(19) United States
(12) Patent Application Publication
(10) Pub. No.: US 2010/0227916 A1
(43) Pub. Date: Sep. 9, 2010

(54) N-DESMETHYL-DOXEPIN AND METHODS OF USING THE SAME TO TREAT SLEEP DISORDERS

(75) Inventors: Neil B. Kavey, Chappaqua, NY (US); Susan E. Dube, Carlsbad, CA (US)

Correspondence Address: KNOBBE MARTENS OLSON & BEAR LLP, 2040 MAIN STREET, FOURTEENTH FLOOR, IRVINE, CA, 92614, US

(73) Assignee: SOMAXON PHARMACEUTICALS, INC, SAN DIEGO, CA (US)

(21) Appl. No.: 12/301,457

(22) PCT Filed: May 18, 2007
(86) PCT No.: PCT/US07/11893

§ 371 Date: Apr. 12, 2010

Related U.S. Application Data
(60) Provisional application No. 60/801,814, filed on May 19, 2006.

(51) Int. Cl. A61K 31/335; C07D 313/12; A61P 25/00

(52) U.S. Cl. 514/450; 549/354

(57) ABSTRACT

The invention relates to desmethyldoxepin, isomers of desmethyldoxepin, and pharmaceutically acceptable salts and prodrugs of desmethyldoxepin; compositions containing the same, and the use of any of the aforementioned for the treatment of sleep disorders.

### BACKGROUND OF THE INVENTION

1. Field of the Invention

The invention relates to desmethyldoxepin, isomers of desmethyldoxepin, and pharmaceutically acceptable salts and prodrugs of desmethyldoxepin; compositions containing the same, and the use of any of the aforementioned for the treatment of sleep disorders.

2. Description of the Related Art

Sleep is essential for health and quality of life. Insomnia is a subjective complaint of dissatisfaction with the quantity, quality or timing of sleep. Insomnia is estimated to occur in approximately 12% to 25% of the general population, although this is probably an underestimate as there is evidence that many adults do not report their sleep problems to a health care professional.

One study has found that fewer than 15% of those who suffer from insomnia are treated with prescription medications. Medications commonly used to treat insomnia include sedative antidepressants, antihistamines, benzodiazepines, and non-benzodiazepine hypnotics. A side effect of some hypnotics is to reduce slow wave sleep. Other side effects of concern are possible daytime residual effects related to sedation, rebound insomnia, and minor side effects specific to each drug class. Tolerance to beneficial effects on sleep is thought to occur with antihistamines and benzodiazepine and non-benzodiazepine hypnotics.

Until the arrival of the non-benzodiazepine hypnotics in the mid '90's, benzodiazepines were the most common drugs used for the pharmacological management of insomnia. These drugs work by binding to and activating sites on the GABA-A receptor complex. Short, intermediate and long-acting benzodiazepines such as triazolam, temazepam and flurazepam were all commonly prescribed for this indication. While these agents have proven to be efficacious and relatively safe, benzodiazepines are associated with a multitude of adverse effects, including residual daytime sedation (“hangover”), amnesia, memory loss and respiratory depression. Rebound insomnia has also been associated with benzodiazepines. Tolerance to the hypnotic effects of the benzodiazepines is common and abrupt discontinuation can result in withdrawal symptoms such as agitation, perceptual changes, confusion, disorientation and insomnia.

Most recently non-benzodiazepine hypnotics have become the primary class of medications for the treatment of insomnia. The leading approved non-benzodiazepine insomnia medications, eszopiclone, zolpidem, and zaleplon, also work by binding to and activating the GABA-A receptors, but they are more selective in their binding than the benzodiazepines. All these drugs approved for the treatment of insomnia that act via the GABA-A receptor, including benzodiazepine and non-benzodiazepine hypnotics, have a potential for addiction and abuse and are classified as Schedule IV controlled substances by the U.S. Drug Enforcement Administration. As a result, many physicians are reluctant to prescribe, and patients are reluctant to take, these drugs for chronic use in treating insomnia. The prescribing of a Schedule IV controlled substance brings scrutiny from the Drug Enforcement Administration and other regulatory bodies, and requires registration and administrative controls in physicians' offices. Therefore, it is desirable to have a pharmacological agent for the treatment of insomnia which is more effective and/or has fewer side effects that those currently used.

Recently a new hypnotic with a mode of action different from other hypnotics has been introduced. Ramelteon is a melatonin receptor agonist with high affinity for melatonin MT1 and MT2 receptors. It is indicated for sleep onset insomnia but it has not been shown to produce a sleep maintenance benefit. It does not affect the GABA-A receptor complex, is not addicting and is not scheduled.

The sedative antidepressants account for a large percentage of the total prescriptions written for insomnia. The National Disease and Therapeutic Index estimates that more than 60% of the 13 million annual trazodone prescriptions are written for the treatment of insomnia, even though trazodone is not indicated for that usage and has never been promoted for that condition. Even though there are very limited data to support the use of trazodone for insomnia and it is associated with undesirable side effects, trazodone is often prescribed because it is a non-scheduled agent, meaning non-addictive, unlike the benzodiazepines and other GABA-receptor agonists which are approved for the treatment of insomnia.

### SUMMARY OF THE INVENTION

Preferred embodiments provide a method for treating insomnia comprising administering to a patient desmethyldoxepin, isomers of desmethyldoxepin, a pharmaceutically acceptable salt thereof, or prodrugs thereof other than doxepin. The administered substance can be delivered, for example, in a daily dosage ranging from about 0.01 to about 500 milligrams. In some embodiments, the daily dosage can range, for example, from about 0.1 to about 300 milligrams, from about 20 to about 300 milligrams, from about 0.1 to about 20 milligrams, from about 0.1 to about 10 milligrams, from about 0.1 to about 5 milligrams, or the like. The pharmaceutically acceptable salt can be any salt, including for example the hydrochloride salt. Also, the prodrug can be any prodrug with the exception of doxepin. For example, the prodrug can be a prodrug ester, amide and the like.

In some aspects the insomnia can be a chronic insomnia or a non-chronic insomnia. For chronic (e.g., greater than 3-4 weeks) or non-chronic insomnias, a patient may suffer from difficulties in sleep onset, sleep maintenance (interruption of sleep during the night by periods of wakefulness), sleep duration, sleep efficiency, premature early-morning awakening, or a combination thereof. Also, the insomnia may be attributable to the concurrent use of other medication, for example. The non-chronic insomnia can be, for example, a short term insomnia or a transient insomnia. The chronic or non-chronic insomnia can be a primary insomnia or an insomnia that is secondary to another condition, for example a disease such as depression or chronic fatigue syndrome. In some aspects, the patient can one that is not suffering from an insomnia that is a component of a disease, or a patient can be treated that is otherwise healthy. As previously mentioned, the chronic or non-chronic insomnia can be a primary insomnia, that is, one that is not attributable to another mental disorder, a general medical condition, or a substance. In many cases, such conditions may be associated with a chronic insomnia and can include, but are not limited to, insomnia attributable to a diagnosable DSM-IV disorder, a disorder such as anxiety or depression, or a disturbance of the physiological sleep-wake system. In some aspects the insomnia can be non-chronic, or of short duration (e.g., less than 3-4 weeks). Examples of causes of such insomnia may be extrinsic or intrinsic and include, but are not limited to environmental sleep disorders as defined by the International Classification of Sleep Disorders (ICSD) such as inadequate sleep hygiene, altitude insomnia or adjustment sleep disorder (e.g., bereavement). Also, short-term insomnia may also be caused by disturbances such as shift-work sleep disorder.

In some embodiments, the insomnia can be treated by administering a trans-(E) or a cis-(Z) isomer of desmethyldoxepin. In still other embodiments, a mixture of the trans-(E) and cis-(Z) isomers can be administered. In some embodiments, the mixture can include an (E/Z) ratio wherein the amount of the Z isomer is greater than the amount of the E isomer. For example, the ratio can be about 1:99, 2:98, 5:95, 10:90, about 15:85, about 25:75, about 30:70, or about 40:60. In some aspects the desmethyldoxepin can be administered in an amount with greater than 25%, 30%, 50%, or 75%, for example, cis-(Z) isomer. In some embodiments, the mixture can include an (E/Z) ratio wherein the amount of the E isomer is greater than the amount of the Z isomer. For example, the ratio can be about 99:1, 98:2, 95:5, 90:10, about 85:15, about 75:25, about 70:30, or about 60:40.

Also, some embodiments relate to a composition that includes desmethyldoxepin, pharmaceutically acceptable salts thereof, and/or prodrugs thereof other than doxepin. In a preferred embodiment, such composition of desmethyldoxepin, a non-doxepin prodrug thereof, or a pharmaceutically acceptable salt thereof can be provided in a daily dosage and/or unit dosage ranging from about 0.01 to about 500 milligrams.

The pharmaceutically acceptable salt of desmethyldoxepin can be the hydrochloride salt, for example.

Also, some embodiments relate to compositions that include desmethyldoxepin, a pharmaceutically acceptable salt of desmethyldoxepin, or a prodrug of desmethyldoxepin other than doxepin, and the composition further can include a pharmaceutically acceptable carrier. In some aspects, the compositions can include desmethyldoxepin, a pharmaceutically acceptable salt thereof, or a prodrug of desmethyldoxepin other than doxepin, in a daily dosage that can range from about 0.01 to about 500 milligrams, about 20 to about 300 milligrams, about 0.1 to about 20 milligrams, about 0.1 to about 10 milligrams, or about 0.1 to about 5 milligrams, for example. Also, the desmethyldoxepin can include greater than 25%, 30%, 50% or 75% cis-(Z) isomer, for example. Also, the composition can include a mixture of E and Z isomers in an E/Z ratio, for example, of 1:99, 2:98, 5:95, 10:90, about 15:85, about 25:75, about 30:70, or about 40:60. In other aspects, the composition can include a mixture of E and Z isomers in an E/Z ratio of 99:1, 98:2, 95:5, 90:10, about 85:15, about 75:25, about 70:30, or about 60:40, for example. In some aspects the compositions can include, for example, 99.5%, 99.9% or 100% of the E or the Z isomer.

Still some embodiments relate to a composition for oral or nasal administration that includes desmethyldoxepin, a pharmaceutically acceptable salt thereof, or a prodrug of desmethyldoxepin other than doxepin, in a dose ranging from about 0.01 to about 500 milligrams. In some aspects the dose can be about 20 to about 300 milligrams, about 0.1 to about 20 milligrams, about 0.1 to about 10 milligrams, or about 0.1 to about 5 milligrams, for example. The pharmaceutically acceptable salt of desmethyldoxepin can be, for example, the hydrochloride salt. Also, the desmethyldoxepin can include, for example, greater than 25%, 30%, 50% or 75% cis-(Z) isomer, or at least 75% cis-(Z) isomer. Also, the composition can include a mixture of E and Z isomers in an E/Z ratio, for example, of 1:99, 2:98, 5:95, 10:90, about 15:85, about 25:75, about 30:70, or about 40:60. In other aspects, the composition can include a mixture of E and Z isomers in an E/Z ratio of 99:1, 98:2, 95:5, 90:10, about 85:15, about 75:25, about 70:30, or about 60:40, for example. In some aspects the compositions can include, for example, 99.5%, 99.9% or 100% of the E or the Z isomer.

Some embodiments relate to methods for treating insomnia, which methods can include, for example, administering to a patient an isomer of desmethyldoxepin, a pharmaceutically acceptable salt of the isomer, or a prodrug of the isomer other than doxepin, in a daily dosage ranging from about 0.01 to about 500 milligrams. In some aspects, about 99.5%, 99.9%, or 100% cis-(Z) isomer can be administered, for example. In other aspects, about 99.5%, 99.9%, or 100% trans-(E) isomer can be administered, for example. Also, the isomer of desmethyldoxepin, or the salt or the prodrug of desmethyldoxepin can be administered in a cis-(Z) to trans-(E) isomer ratio of about 99:1, 98:2, 95:5, 90:10, 80:20, 70:30, 60:40, 50:50, 40:60, 30:70, 20:80, 10:90, 5:95, 2:98 or 1:99. Preferably, the isomer of desmethyldoxepin, the salt or the prodrug is greater than 25% or 50% cis-(Z) isomer. The insomnia can be a chronic or a non-chronic insomnia. For example, the non-chronic insomnia can be a transient or a short term insomnia. Also, the insomnia can be an onset insomnia or a maintenance insomnia.

Some embodiments provide for a use of a compound in the preparation of a medicament for the treatment of insomnia, said compound being desmethyldoxepin and pharmaceutically acceptable salts and pro-drugs thereof other than doxepin, in a daily dosage that can range from about 0.01 to about 500 milligrams. In some embodiments the pharmaceutically acceptable salt can be a hydrochloride salt. In some embodiments, the prodrug of desmethyldoxepin can be a prodrug ester, amide and the like.

In some aspects, the use of desmethyldoxepin, a pharmaceutically acceptable salt thereof, or a prodrug of desmethyldoxepin other than doxepin to prepare a medicament for the treatment of insomnia can be in a daily dosage that can range from about 0.01 to about 500 milligrams, about 20 to about 300 milligrams, about 0.1 to about 20 milligrams, about 0.1 to about 10 milligrams, or about 0.1 to about 5 milligrams, for example. Also, the desmethyldoxepin can include greater than 25%, 30%, 50% or 75% cis-(Z) isomer, for example. Also, the medicament can include a mixture of E and Z isomers of desmethyldoxepin, pharmaceutically acceptable salt, or a prodrug thereof in an E/Z ratio, for example, of 1:99, 2:98, 5:95, 10:90, about 15:85, about 25:75, about 30:70, or about 40:60. In other aspects, the medicament can include a mixture of E and Z isomers of desmethyldoxepin in an E/Z ratio of 99:1, 98:2, 95:5, 90:10, about 85:15, about 75:25, about 70:30, or about 60:40, for example. In some aspects the medicament can include, for example, 99.5%, 99.9% or 100% of the E or the Z isomer.

In some aspects, the medicament can include desmethyldoxepin, a pharmaceutically acceptable salt thereof, or a prodrug of desmethyldoxepin other than doxepin, and be given in a daily dosage that can range from about 0.01 to about 500 milligrams, about 20 to about 300 milligrams, about 0.1 to about 20 milligrams, about 0.1 to about 10 milligrams, or about 0.1 to about 5 milligrams, for example.

In some embodiments, the medicament can be used to treat a chronic insomnia or a non-chronic insomnia. For example, the non-chronic insomnia can a transient or a short term insomnia. Additionally, the insomnia can be onset insomnia or maintenance insomnia.

### DETAILED DESCRIPTION OF THE PREFERRED EMBODIMENT

Generally, embodiments of the present invention relate to the use of desmethyldoxepin to treat an individual having a sleep disorder, such as, for example, insomnia. In some embodiments, the sleep disorder can be treated by administering desmethyldoxepin in a low dosage, while in others it can be administered in a higher dosage. In some embodiments, a single isomer of desmethyldoxepin can be used or a particular ratio of isomers can be used. Also, in some embodiments a pharmaceutically acceptable salt of desmethyldoxepin can be used. Furthermore, in some embodiments a prodrug of desmethyldoxepin other than doxepin can be used. U.S. Application No. 60/801,814, filed May 19, 2006 is incorporated herein by reference in its entirety.

Transient insomnia is an insomnia that is present for one to several days, and is less than one week in duration. Short term insomnia is insomnia of one to three or four weeks in duration. Chronic insomnia is typically accepted to involve episodes greater than three (3) or four (4) weeks in duration.

Insomnia may further involve difficulty in falling asleep, referred to as onset insomnia and/or difficulty in maintaining uninterrupted sleep, referred to as maintenance insomnia. It is well known that the sleep deprivation resulting from such insomnia adversely affects cognition, safety and quality of life. Even in otherwise healthy young people, sleep deprivation has been associated, for example, with changes in body physiology such as changes in thyroid function, changes in glucose metabolism and insulin resistance.

Desmethyldoxepin is also known as nordoxepin and has the following structure:

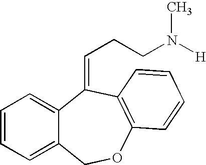

Desmethyldoxepin is commercially available as a forensic standard. For example, it can be obtained from Cambridge Isotope Laboratories, Inc. (50 Frontage Road, Andover, Mass. Desmethyldoxepin for use in the methods discussed herein can be prepared by any suitable procedure. For example, desmethyldoxepin can be prepared from 3-methylaminopropyl triphenylphosphonium bromide hydrobromide and 6,11-dihydrodibenz(b,e)oxepin-11-one according to the method taught in U.S. Pat. No. 3,509,175, which is incorporated herein by reference in its entirety. As another example, desmethyldoxepin can be prepared in its (E) and (Z) isomers from doxepin hydrochloride as taught in U.S. Pat. No. 5,332,661, which is incorporated herein by reference in its entirety.

As mentioned above, in addition to desmethyldoxepin, the methods and other embodiments described herein can utilize any suitable pharmaceutically acceptable salt or prodrug of desmethyldoxepin or an isomer of desmethyldoxepin other than doxepin (or its isomers). Therefore, the substitution or use in combination of salts and prodrugs is specifically contemplated in the various embodiments, even though, only desmethyldoxepin may be specifically mentioned. The pharmaceutically acceptable salts and prodrugs can be made by any suitable method. The acids that may be used to prepare pharmaceutically acceptable acid addition salts of desmethyldoxepin are those that form non-toxic acid addition salts, i.e., salts containing pharmacologically acceptable anions, such as the acetate, benzenesulfonate, benzoate, bicarbonate, bisulfate, bitartrate, borate, bromide, calcium edetate, camsylate, carbonate, chloride, clavulanate, citrate, dihydrochloride, edetate, dislyate, estolate, esylate, ethylsuccinate, fumarate, gluceptate, gluconate, glutamate, glycollylarsanilate, hexylresorcinate, hydrabamine, hydrobromide, hydrochloride, hydroiodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phospate/diphosphate, polygalacturonate, salicylate, stearate, subacetate, succinate, tannate, tartrate, teoclate, tosylate, triethiodode, and valerate salts. The term “prodrug” refers to a chemical entity that is rapidly transformed in vivo to yield an active entity, for example, desmethyldoxepin, such as by hydrolysis in blood or inside tissues, for example. As mentioned above doxepin is specifically excluded from the prodrug embodiments described herein. Examples of prodrug groups can be found in, for example, T. Higuchi and V. Stella, in “Pro-drugs as Novel Delivery Systems,” Vol. 14, A.C.S. Symposium Series, American Chemical Society (1975); H. Bundgaard, “Design of Prodrugs,” Elsevier Science, 1985; and “Bioreversible Carriers in Drug Design: Theory and Application,” edited by E. B. Roche, Pergamon Press: New York, 14-21 (1987), each of which is hereby incorporated by reference in its entirety.

One example of a suitable prodrug is a prodrug amide. Prodrug amides can hydrolyze under physiological conditions to afford a free amine, such as desmethyldoxepin, and a carboxylic acid (Hellberg, et al., “The Hydrolysis of the Prostaglandin Analog Prodrug Bimatoprost to 17-Phenyl-trinor PGF2 by Human and Rabbit Ocular Tissue”, 2003, 19 (2), 97-103), which is incorporated herein by reference in its entirety.

In some embodiments, the sleep disorder can be treated, for example, by administering an isomer of desmethyldoxepin. For example, a single isomer can be administered or the sleep disorder can be treated by administering a mixture of the trans-(E) and cis-(Z) isomers of desmethyldoxepin. In some preferred embodiments a higher ratio of the cis-(Z) isomer can be administered. For example, preferably the ratio of E/Z isomers administered can be about 1/99, about 2/98, about 3/97, about 4/96, about 5/95, about 6/94, about 7/93, about 8/92, about 9/91 or about 10/90. Also, in some embodiments the ratio can be about 15/85, about 20/80, or about 30/70. In some embodiments the ratio of E/Z can be about 40/60, about 50/50 and the like. Also, in some embodiments, the ratio of E/Z isomers administered can be about 99/1, about 98/2, about 97/3, about 96/4, about 95/5, about 94/6, about 93/7, about 92/8, about 91/9 or about 90/10, for example. In some embodiments the ratio of E/Z isomers can be about 85/15, about 80/20, about 70/30, about 60/40, about 50/50 and the like. In some aspects, the ratio of E/Z isomers administered can vary from about 5/95 to about 40/60, from about 15/85 to about 35/65, from about 20/80 to about 30/70, from about 95/5 to about 60/40, from about 85/15 to about 65/35, from about 80/20 to about 70/30, or about 50/50, or the like. (E)-desmethyldoxepin and (Z)-desmethyldoxepin can be prepared from doxepin hydrochloride as taught in the incorporated material of U.S. Pat. No. 5,332,661.

Desmethyldoxepin is a metabolite of doxepin in which a methyl group is removed from the amino group of doxepin. Doxepin belongs to a class of psychotherapeutic agents known as dibenzoxepin tricyclic compounds, and is currently approved and prescribed for use as an antidepressant to treat depression and anxiety. Doxepin has a well-established safety profile, having been prescribed for over 35 years. For example, doxepin is currently prescribed for a depressed patient population in typical dosages varying from about 75 to about 300 milligrams per day. The use of low dosage of doxepin for treatment of a patient suffering from chronic insomnia and transient or short term insomnia is discussed in U.S. Pat. Nos. 5,502,047 and 6,211,229, respectively, both are herein incorporated by reference.

Unlike doxepin, desmethyldoxepin is not currently approved for any indication. Surprisingly, desmethyldoxepin is very effective in treating insomnia, has little or no abuse potential, has a rapid onset of action, and very minimal side effects when used in low doses. Prior to the present invention, very little was known about any sedative or hypnotic effects of desmethyldoxepin. In addition, in view of its relatively long half life, it is surprising that desmethyldoxepin can be used to treat insomnia with little or no hangover effect, particularly at lower doses.

### Insomnia

Some embodiments relate to the use of desmethyldoxepin, an isomer or a particular ratio of isomers, pharmaceutically acceptable salts, and/or prodrugs in the treatment of chronic and non-chronic insomnia. Examples of non-chronic insomnia include, for example, transient insomnia and short-term insomnia. Transient insomnia is an insomnia that is present for about one to several days, and is less than one week in duration. Short term insomnia is insomnia of about one to three weeks or four weeks in duration. Chronic insomnia is typically accepted to involve episodes greater than three (3) or four (4) weeks in duration. It is well known that the sleep deprivation resulting from such insomnia adversely affects cognition, safety and quality of life.

Furthermore, for chronic (e.g., greater than 3-4 weeks) or non-chronic insomnias, a patient may suffer from difficulties in sleep onset, sleep maintenance (interruption of sleep during the night by periods of wakefulness), sleep duration, sleep efficiency, premature early-morning awakening, or a combination thereof. Also, the insomnia may be attributable to the concurrent use of other medication, for example.

The chronic or non-chronic insomnia can be a primary insomnia or an insomnia that is secondary or attributable to another condition, for example a disease such as depression or chronic fatigue syndrome. In some aspects, the patient can be one that is not suffering from an insomnia that is a component of a disease. In some aspects, the methods can specifically exclude a patient with a secondary insomnia, for example, a patient suffering from insomnia as a component of depression or chronic fatigue syndrome. Some embodiments relate to methods of treating individuals suffering from insomnia that is caused by injury or the use of a medication or other substance. Treating such patients can specifically be excluded from other methods of treatment.

As previously mentioned, the chronic or non-chronic insomnia can be a primary insomnia, that is, one that is not attributable to another mental disorder, a general medical condition, or a substance. In many cases, such conditions may be associated with a chronic insomnia and can include, but are not limited to, insomnia attributable to a diagnosable DSM-IV disorder, a disorder such as anxiety or depression, or a disturbance of the physiological sleep-wake system. The non-chronic or short duration insomnia (e.g., less than 3-4 weeks) can have intrinsic or extrinsic causes. For example, non-chronic sleep disorders can include, but are not limited to, environmental sleep disorders as defined by the International Classification of Sleep Disorders (ICSD) such as inadequate sleep hygiene, altitude insomnia or adjustment sleep disorder (e.g., bereavement). Also, short-term insomnia may also be caused by disturbances such as shift-work sleep disorder.

In some embodiments, an otherwise healthy individual can be treated for insomnia. For example, desmethyldoxepin can be used to treat an individual suffering from an insomnia that is not attributable to a medical, psychiatric, or environmental cause. In some embodiments, methods of treating otherwise healthy individual can be specifically excluded from the methods.

In some embodiments an individual having a secondary insomnia, for example, insomnia as a component of his/her depression or other illness, can be treated, while in others methods of treatment of such individuals can be specifically excluded. Also, in some embodiments, an individual suffering from insomnia as part of chronic fatigue syndrome can be treated, while in other embodiments such the treatment of such individuals is excluded. Some embodiments relate to methods of treating individuals suffering from insomnia that is caused by injury or the use of a medication or other substance. Treating such patients can specifically be excluded from other methods of treatment.

The desmethyldoxepin can be administered and dosed as described below.

### Pharmaceutical Compositions and Administration

As discussed above, desmethyldoxepin, isomers, pharmaceutically acceptable salts, prodrugs and compositions that include any of the same can be used to treat insomnia in a mammal, including a human. Methods of use can include the step of administering a therapeutically effective amount of the compound to a mammal in need thereof.

Actual dosage levels of the compounds in the pharmaceutical compositions may be varied so as to administer an amount of the compound that is effective to achieve the desired therapeutic response for a particular patient. Examples of dosages that can be used are described more fully elsewhere herein.

Suitable routes of administration include oral, buccal, sublingual; transdermal, rectal, topical, transmucosal, or intestinal administration; parenteral delivery, including intramuscular, subcutaneous, intravenous, intramedullary injections, as well as intrathecal, direct intraventricular, intraperitoneal, intranasal, or intraocular injections.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Administration though oral pathways can be accomplished, for example, using a capsule, a tablet, a granule, a spray, a syrup, a liquid, powder, granules, pastes (e.g., for application to the tongue). Oral administration can be accomplished using fast-melt formulations, for example. Pharmaceutical preparations for oral use can be obtained by mixing one or more solid excipient with pharmaceutical combination of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). For low dose formulations excipients such as Starch 1500® and the like can be useful. If desired, disintegrating agents may be added, such as the cross-linked polyvinyl pyrrolidone, agar, or alginic acid or a salt thereof such as sodium alginate.

Pharmaceutical preparations which can be used orally, including sublingually, include for example, liquid solutions, powders, and suspensions in bulk or unit dosage forms. Also, the oral formulations can include, for example, pills, tablets, granules, sprays, syrups, pastes, powders, boluses, pre-measured ampules or syringes, push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take any suitable form, for example, tablets or lozenges.

For topical administration, the compounds may be formulated for administration to the epidermis as ointments, gels, creams, pastes, salves, gels, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition, any of the compounds and compositions described herein can also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. Furthermore, any of the compounds and compositions described herein also can be formulated as a fast-melt preparation. The compounds and compositions can also be formulated and administered as a drip, a suppository, a salve, an ointment, an absorbable material such a transdermal patch, or the like.

One can also administer the compounds of the invention in sustained release forms or from sustained release drug delivery systems. A description of representative sustained release materials can be found in the incorporated materials in Remington: The Science and Practice of Pharmacy (20^{th} ed, Lippincott Williams & Wilkens Publishers (2003)), which is incorporated herein by reference in its entirety.

A variety of techniques for formulation and administration can be found in Remington: The Science and Practice of Pharmacy (20^{th} ed, Lippincott Williams & Wilkens Publishers (2003)), which is incorporated herein by reference in its entirety.

Desmethyldoxepin, its isomers alone or in a ratio, pharmaceutically acceptable salts, and/or prodrugs can be used alone or in combination with other substances, such as for example, other insomnia or sleep medications, or with other medications that treat a primary illness. The desmethyldoxepin alone or in combination can be included as part of a composition. The compounds and compositions can include any suitable form of the compound for pharmaceutical delivery, as discussed in further detail herein. For example, in certain embodiments, the compounds or compositions comprising the same may include a pharmaceutically acceptable salt of the compound.

The compositions and formulations disclosed herein also can include one or more pharmaceutically acceptable carrier materials or excipients. Such compositions can be prepared for storage and for subsequent administration. Acceptable carriers or diluents for therapeutic use are well known in the pharmaceutical art, and are described, for example, in the incorporated material of Remington: The Science and Practice of Pharmacy (20^{th} ed, Lippincott Williams & Wilkens Publishers (2003)), which is incorporated herein by reference in its entirety. The term “carrier” material or “excipient” herein can mean any substance, not itself a therapeutic agent, used as a carrier and/or diluent and/or adjuvant, or vehicle for delivery of a therapeutic agent to a subject or added to a pharmaceutical composition to improve its handling or storage properties or to permit or facilitate formation of a dose unit of the composition into a discrete article such as a capsule or tablet suitable for oral administration. Excipients can include, by way of illustration and not limitation, diluents, disintegrants, binding agents, adhesives, wetting agents, polymers, lubricants, glidants, substances added to mask or counteract a disagreeable taste or odor, flavors, dyes, fragrances, and substances added to improve appearance of the composition. Acceptable excipients include lactose, sucrose, starch powder, maize starch or derivatives thereof, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinyl-pyrrolidone, and/or polyvinyl alcohol, saline, dextrose, mannitol, lactose, lecithin, albumin, sodium glutamate, cysteine hydrochloride, and the like. Examples of suitable excipients for soft gelatin capsules include vegetable oils, waxes, fats, semisolid and liquid polyols. Suitable excipients for the preparation of solutions and syrups include, without limitation, water, polyols, sucrose, invert sugar and glucose. Suitable excipients for injectable solutions include, without limitation, water, alcohols, polyols, glycerol, and vegetable oils. The pharmaceutical compositions can additionally include preservatives, solubilizers, stabilizers, wetting agents, emulsifiers, sweeteners, colorants, flavorings, buffers, coating agents, or antioxidants. Sterile compositions for injection can be formulated according to conventional pharmaceutical practice as described in the incorporated material in Remington: The Science and Practice of Pharmacy (20^{th} ed, Lippincott Williams & Wilkens Publishers (2003)). For example, dissolution or suspension of the active compound in a vehicle such as water or naturally occurring vegetable oil like sesame, peanut, or cottonseed oil or a synthetic fatty vehicle like ethyl oleate or the like may be desired. Buffers, preservatives, antioxidants and the like can be incorporated according to accepted pharmaceutical practice. The compound can also be made in microencapsulated form. In addition, if desired, the injectable pharmaceutical compositions may contain minor amounts of nontoxic auxiliary substances, such as wetting agents, pH buffering agents, and the like. If desired, absorption enhancing preparations (for example, liposomes), can be utilized.

The compositions and formulations can include any other agents that provide improved transfer, delivery, tolerance, and the like. These compositions and formulations can include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol. Pharmacol. 32 (2):210-8 (2000), Charman W N “Lipids, lipophilic drugs, and oral drug delivery-some emerging concepts.” J Pharm Sci. 89 (8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

The selected dosage level can depend upon, for example, the route of administration, the severity of the condition being treated, and the condition and prior medical history of the patient being treated. However, it is within the skill of the art to start doses of the compound at levels lower than required to achieve the desired therapeutic effect and to gradually increase the dosage until the desired effect is achieved. It will be understood, however, that the specific dose level for any particular patient can depend upon a variety of factors including the genetic makeup, body weight, general health, diet, time and route of administration, combination with other drugs and the particular condition being treated, and its severity. For the treatment of insomnia, preferably one dose is administered prior to bedtime.

### Dosages

Any suitable dosage of desmethyldoxepin, isomer of desmethyldoxepin, a pharmaceutical salt, or prodrug can be used to treat the sleep disorder such as insomnia. In some aspects, daily dosages of desmethyldoxepin, isomer, pharmaceutically acceptable salt, or prodrug may vary from about 0.01 to about 500 milligrams, from about 0.01 to about 300 milligrams, from about 0.05 to about 300 milligrams, from about 0.1 to about 300 milligrams, from about 1 to about 200 milligrams, or from about 5, 10 or 20 milligrams to about 300 milligrams. Preferably, daily dosages of about 10, about 20, about 50, about 75 milligrams or less can utilized. In other aspects, a daily dosage of greater than about 10, about 20, about 50, or about 75 milligrams can be used. However, as it is recognized that each individual may react differently to a given dose of the medication used, the dosages recited should be accorded flexibility. Further, any suitable unit dosage form can be formulated to contain desmethyldoxepin, an isomer, a prodrug or a pharmaceutically acceptable salt in the above-recited amounts (e.g., 0.01-500 mg) or greater.

In general, lower doses of desmethyldoxepin are preferred. These low doses are surprisingly effective and, in most patients, have almost no side effects. In some embodiments, daily dosages of desmethyldoxepin can be about 0.01, 0.02, 0.03, 0.04, 0.05, 0.06, 0.07, 0.08 or 0.09 milligrams. In some embodiments about 0.1, 0.2, 0.3, 0.4, 0.5, 0.6, 0.7, 0.8, 0.9, or 1.0 milligrams of desmethyldoxepin can be used. In another embodiment, daily dosages of desmethyldoxepin may be about 1, 2, 3, 4, 5, 6, 7, 8, 9, or 10 milligrams. In another embodiment, daily dosages of desmethyldoxepin may be about 11, 12, 13, 14, 15, 16, 17, 18, 19, or 20 milligrams. Therapy at each of the doses described in this paragraph as well as ranges between these doses, are particularly contemplated. These relatively low doses between 0.01 milligrams up to, for example, 2, 3, 4, 5, 10, 15 or 20 milligrams, have reduced side effects, are surprisingly effective, and have a relatively rapid onset.

Further, in some embodiments, where patients are in need of more drug than is provided by the low-dose therapy described above, daily dosages of desmethyldoxepin may be up to about 25 or 30 milligrams. In another embodiment, daily dosages of desmethyldoxepin may be up to about 35 or 40 milligrams. In another embodiment, daily dosages of desmethyldoxepin may be up to about 45 or 50 milligrams.

In some embodiments, daily dosages of desmethyldoxepin can range up to about 50 milligrams to about 100 milligrams, about 50 to 59 milligrams, about 60-69 milligrams, about 70-79 milligrams, about 80-89 milligrams, or about 90-100 milligrams. In some embodiments, daily dosages of desmethyldoxepin can be about 100 to 500 milligrams, about 100 to about 300 milligrams, 100 to about 150 milligrams, 151 to about 200 milligrams, 201 to about 250 milligrams, or about 251 to about 300 milligrams. In some embodiments, desmethyldoxepin can be administered in a daily dosage of about 300 to about 500 milligrams, or more.

### EXAMPLES

### Example 1

Desmethyldoxepin is prepared according to the following method. Anhydrous 3-methylaminopropyltriphenylphosphonium bromide hydrobromide (1530 g) prepared as in U.S. Pat. No. 3,509,175, is suspended in 4.5 L dry tetrahydrofuran and 6.0 moles of butyl lithium in heptane is added during 1 hour. After an additional 30 minutes, 483 g of 6,11-dihydrodibenz(b,e)oxepin-11-one, is added to the deep red solution and the reaction is maintained at reflux for 10 hours. Water, 500 mL, is added at room temperature and the solvent is removed in vacuo. The crude residue is treated with 10% hydrochloric acid until acidic (pH 2) and then 1.5 L benzene is added. After stirring, the mixture separates into three phases (an insoluble hydrochloride salt product phase, an aqueous phase and an organic phase). The benzene layer is removed by decantation and the remaining mixture is rendered basic with 10% sodium hydroxide solution and is extracted with 3×1500 mL portions of benzene. The benzene extracts are washed, then dried with anhydrous sodium sulfate and concentrated in a vacuum leaving a solid residue of desmethyldoxepin.

### Example 2

(E)-Desmethyldoxepin is prepared from doxepin hydrochloride as follows. Doxepin hydrochloride (E/Z=85/15) (55.0 g, 0.174 mol) is dissolved in 600 mL H₂O, made basic with 6M NaOH, and extracted with CHCl₃ (3×600 mL). The CHCl₃ extracts are combined, dried over Na₂SO₄, and solvent removed in vacuo. The resulting oil is dissolved in 250 mL EtOH, then 21.15 g (0.182 mol) of maleic acid dissolved in 100 mL EtOH is added slowly, with stirring, followed by an additional 350 mL EtOH. The resulting cloudy solution is refluxed until it becomes clear, then allowed to stand overnight at room temperature; the resulting crystals are isolated by vacuum filtration. Additional recrystallization from EtOH yields a white crystalline product ((E)-Doxepin maleate) with an E/Z ratio of 98/2. (E)-Doxepin maleate (2.50 g, 6.32 mmol) is then partially dissolved in 60 mL H₂O, made basic with 6M NaOH, and extracted with CHCl₃ (3×60 mL). The CHCl₃ extracts are combined, washed with 60 mL brine, dried over Na2SO4, and solvent removed in vacuo. The resulting oil is re-dissolved in 10 mL CHCl₃, 1.8 mL (13 mmol) of triethylamine added, 1.8 mL (13 mmol) of 2,2,2-trichloroethylchloro-formate added, and reaction stirred under N₂ for 3.5 hours. The completed reaction is then diluted with 140 mL Et₂O, washed successively with 0.5M HCl (2×140 mL), H₂O (140 mL), and brine (140 mL), then dried over MgSO₄ and solvent removed in vacuo. Resulting material is further purified by silica gel column chromatography, eluting with EtOAc/Hex (20/80), to afford 1.48 g (3.36 mmol) of the desired product as a clear oil. The N-protected (E)-desmethyldoxepin intermediate (1.44 g, 3.27 mmol) is then dissolved in 12 mL THF, 2.88 g of zinc powder added, 2.3 mL of 1M sodium phosphate (pH=5.5) added, and reaction stirred for 17 hours. The reaction mixture is then vacuum filtered, filtrate solvent removed in vacuo, and resulting residue purified by silica gel column chromatography, eluting with THF/MeOH/NH₄OH (85/15/0.4), then THF/MeOH/NH₄OH (75/25/0.4), to afford 744 mg (2.80 mmol) of the desired product as a pale yellow solid.

### Example 3

(Z)-Desmethyldoxepin is prepared from doxepin hydrochloride as follows. Doxepin hydrochloride (E/Z=85/15) (100 g, 0.317 mol) is dissolved in 800 mL H₂O, made basic with 6M NaOH, and extracted with CHCl₃ (3×800 mL). The CHCl₃ extracts are combined, dried over Na₂SO₄, and solvent removed in vacuo. The resulting oil is dissolved in 700 mL EtOH, then 36.7 g (0.317 mol) of maleic acid dissolved in 600 mL EtOH is added slowly, with stirring. The resulting cloudy solution is refluxed until clear, then allowed to stand overnight at room temperature. Crystals are isolated by vacuum filtration and the mother liquor saved. Crystals are recrystallized two additional times as above, and the three mother liquors saved and combined and solvent removed in vacuo. Recrystallization of mother liquor material from refluxing EtOH eventually affords 24 g of a mother liquor product which is 65% Z isomer in composition. Recrystallization of this material from 450 mL EtOH gives crystals (9.1 g) which are 80% Z isomer. This material is recrystallized from 170 mL CHCl₃/CCl₄ (50/50) at 4° C., yielding 7.65 g of crystalline material which is 87% Z isomer in composition. Three additional recrystallizations from CHCl₃/CCl₄ eventually affords 5.12 g (12.9 mmol) of the desired product ((Z)-Doxepin maleate) with an E/Z ratio of 4/96; melting point: 162°-163° C. (Z)-Doxepin maleate (1.00 g, 2.53 mmol) is then partially dissolved in 35 mL H₂O, made basic with 6M NaOH, and extracted with CHCl₃ (3×35 mL). The CHCl₃ extracts are combined, washed with 35 mL brine, dried over Na₂SO₄, and solvent removed in vacuo. The resulting oil is re-dissolved in 4 mL CHCl₃, 0.65 mL (4.7 mmol) of triethylamine added, 0.65 mL (4.7 mmol) of 2,2,2-trichloroethyl-chloroformate added, and reaction stirred under N₂ for 3.5 hours. The completed reaction is then diluted with 50 mL Et₂O, washed successively with 0.5M HCl (2×50 mL), H₂O (50 mL), and brine (50 mL), then dried over MgSO₄ and solvent removed in vacuo. Resulting material is further purified by silica gel column chromatography, eluting with EtOAc/Hex (20/80), to afford 710 mg (1.61 mmol) of the desired product as a clear oil. The N-protected (Z)-desmethyldoxepin (679 mg, 1.54 mmol) is then dissolved in 5.7 mL THF, 1.36 g of zinc powder added, 1.1 mL of 1M sodium phosphate (pH=5.5) added, and reaction stirred for 17 hours. The reaction mixture is then vacuum filtered, filtrate solvent removed in vacuo, and resulting residue purified by silica gel column chromatography, eluting with THF/MeOH/NH₄OH (85/15/0.4), then THF/MeOH/NH₄OH (82/18/0.4), to afford 364 mg (1.37 mmol) of the desired product as a pale yellow solid.

### Example 4

A patient suffers from transient or short term insomnia. The patient is otherwise healthy with normal affect with no depression, anxiety or substance overuse. The patient is prescribed desmethyldoxepin in a daily dosage of 1 milligram, prior to bedtime. Administration of desmethyldoxepin relieves the insomnia.

### Example 5

A patient suffers from transient or short term insomnia. The patient is otherwise healthy with normal affect with no depression, anxiety or substance overuse. The patient is prescribed desmethyldoxepin in a daily dosage of 2 milligrams, prior to bedtime. Administration of desmethyldoxepin relieves the insomnia.

### Example 6

A patient suffers from transient or short term insomnia. The patient also suffers from depression. The patient is prescribed desmethyldoxepin in a daily dosage of 2 milligrams, prior to bedtime. Administration of desmethyldoxepin relieves the insomnia.

### Example 7

A patient suffers from transient or short term insomnia. The patient is otherwise healthy with normal affect with no depression, anxiety or substance overuse. The patient is prescribed desmethyldoxepin in a daily dosage of 5 milligrams, prior to bedtime. Administration of desmethyldoxepin relieves the insomnia.

### Example 8

A patient suffers from transient or short term insomnia. The patient also suffers from depression. The patient is prescribed desmethyldoxepin in a daily dosage of 5 milligrams, prior to bedtime. Administration of desmethyldoxepin relieves the insomnia.

### Example 9

A patient suffers from transient or short term insomnia. The patient is otherwise healthy with normal affect with no depression, anxiety or substance overuse. The patient is prescribed desmethyldoxepin in a daily dosage of 10 milligrams, prior to bedtime. Administration of desmethyldoxepin relieves the insomnia.

### Example 10

A patient suffers from transient or short term insomnia. The patient also suffers from depression. The patient is prescribed desmethyldoxepin in a daily dosage of 10 milligrams, prior to bedtime. Administration of desmethyldoxepin relieves the insomnia.

### Example 11

A patient suffers from transient or short term insomnia. The patient is otherwise healthy with normal affect with no depression, anxiety or substance overuse. The patient is prescribed desmethyldoxepin in a daily dosage of 20 milligrams prior to bedtime. Administration of desmethyldoxepin relieves the insomnia.

### Example 12

A patient suffers from transient or short term insomnia. The patient also suffers from depression. The patient is prescribed desmethyldoxepin in a daily dosage of 20 prior to bedtime. Administration of desmethyldoxepin relieves the insomnia.

### Example 13

A patient suffers from transient or short term insomnia. The patient is otherwise healthy with normal affect with no depression, anxiety or substance overuse. The patient is prescribed desmethyldoxepin in a daily dosage of 150 milligrams prior to bedtime. Administration of desmethyldoxepin relieves the insomnia.

### Example 14

A patient suffers from transient or short term insomnia. The patient also suffers from depression. The patient is prescribed desmethyldoxepin in a daily dosage of 150 milligrams prior to bedtime. Administration of desmethyldoxepin relieves the insomnia.

### Example 15

A patient suffers from chronic insomnia. The patient is otherwise healthy with normal affect with no depression, anxiety or substance overuse. The patient is prescribed desmethyldoxepin in a daily dosage of 2 milligrams prior to bedtime. Administration of desmethyldoxepin relieves the insomnia.

### Example 16

A patient suffers from chronic insomnia. The patient also suffers from depression. The patient is prescribed desmethyldoxepin in a daily dosage of 1 milligram prior to bedtime. Administration of desmethyldoxepin relieves the insomnia.

### Example 17

A patient suffers from chronic insomnia. The patient also suffers from depression. The patient is prescribed desmethyldoxepin in a daily dosage of 2 milligrams prior to bedtime. Administration of desmethyldoxepin relieves the insomnia.

### Example 18

A patient suffers from chronic insomnia. The patient is otherwise healthy with normal affect with no depression, anxiety or substance overuse. The patient is prescribed desmethyldoxepin in a daily dosage of 5 milligrams prior to bedtime. Administration of desmethyldoxepin relieves the insomnia.

### Example 19

A patient suffers from chronic insomnia. The patient also suffers from depression. The patient is prescribed desmethyldoxepin in a daily dosage of 5 milligrams prior to bedtime. Administration of desmethyldoxepin relieves the insomnia.

### Example 20

A patient suffers from chronic insomnia. The patient is otherwise healthy with normal affect with no depression, anxiety or substance overuse. The patient is prescribed desmethyldoxepin in a daily dosage of 10 milligrams prior to bedtime. Administration of desmethyldoxepin relieves the insomnia.

### Example 21

A patient suffers from chronic insomnia. The patient also suffers from depression. The patient is prescribed desmethyldoxepin in a daily dosage of 10 milligrams prior to bedtime. Administration of desmethyldoxepin relieves the insomnia.

### Example 22

A patient suffers from chronic insomnia. The patient is otherwise healthy with normal affect with no depression, anxiety or substance overuse. The patient is prescribed desmethyldoxepin in a daily dosage of 20 milligrams prior to bedtime. Administration of desmethyldoxepin relieves the insomnia.

### Example 23

A patient suffers from chronic insomnia. The patient also suffers from depression. The patient is prescribed desmethyldoxepin in a daily dosage of 20 milligrams prior to bedtime. Administration of desmethyldoxepin relieves the insomnia.

### Example 24

A patient suffers from chronic insomnia. The patient is otherwise healthy with normal affect with no depression, anxiety or substance overuse. The patient is prescribed desmethyldoxepin in a daily dosage of 150 milligrams prior to bedtime. Administration of desmethyldoxepin relieves the insomnia.

### Example 25

A patient suffers from chronic insomnia. The patient also suffers from depression. The patient is prescribed desmethyldoxepin in a daily dosage of 150 milligrams prior to bedtime. Administration of desmethyldoxepin relieves the insomnia.

### Example 26

A patient suffers from a sleep disorder. The patient is prescribed an isomer of desmethyldoxepin in a daily dosage of 1 milligram prior to bedtime. Administration of the isomer of desmethyldoxepin relieves the insomnia.

### Example 27

A patient suffers from a sleep disorder. The patient is prescribed an isomer of desmethyldoxepin in a daily dosage of 2 milligrams prior to bedtime. Administration of the isomer of desmethyldoxepin relieves the insomnia.

### Example 28

A patient suffers from a sleep disorder. The patient is prescribed an isomer of desmethyldoxepin in a daily dosage of 5 milligrams prior to bedtime. Administration of the isomer of desmethyldoxepin relieves the insomnia.

### Example 29

A patient suffers from a sleep disorder. The patient is prescribed an isomer of desmethyldoxepin in a daily dosage of 10 milligrams prior to bedtime. Administration of the isomer of desmethyldoxepin relieves the insomnia.

### Example 30

A patient suffers from a sleep disorder. The patient is prescribed a pharmaceutically acceptable salt of desmethyldoxepin in a daily dosage of 2 milligrams prior to bedtime. Administration of the pharmaceutically acceptable salt of desmethyldoxepin relieves the insomnia.

### Example 31

A patient suffers from a sleep disorder. The patient is prescribed a pharmaceutically acceptable salt of desmethyldoxepin in a daily dosage of 5 milligrams prior to bedtime. Administration of the pharmaceutically acceptable salt of desmethyldoxepin relieves the insomnia.

### Example 32

A patient suffers from a sleep disorder. The patient is prescribed a pharmaceutically acceptable salt of desmethyldoxepin in a daily dosage of 10 milligrams prior to bedtime. Administration of the pharmaceutically acceptable salt of desmethyldoxepin relieves the insomnia.

### Example 33

A patient suffers from a sleep disorder. The patient is prescribed a prodrug of desmethyldoxepin other than doxepin in a daily dosage of 1 milligram prior to bedtime. Administration of the prodrug relieves the insomnia.

### Example 34

A patient suffers from a sleep disorder. The patient is prescribed a prodrug of desmethyldoxepin other than doxepin in a daily dosage of 2 milligrams prior to bedtime. Administration of the prodrug relieves the insomnia.

### Example 35

A patient suffers from a sleep disorder. The patient is prescribed a prodrug of desmethyldoxepin other than doxepin in a daily dosage of 5 milligrams prior to bedtime. Administration of the prodrug relieves the insomnia.

### Example 36

A patient suffers from a sleep disorder. The patient is prescribed a prodrug of desmethyldoxepin other than doxepin in a daily dosage of 10 milligrams prior to bedtime. Administration of the prodrug relieves the insomnia.

### Example 37

A patient suffers from a sleep disorder. The patient is prescribed a mixture of E and Z isomers of desmethyldoxepin containing greater than 25% Z isomer in a daily dosage of 1 milligram prior to bedtime. Administration of the isomers of desmethyldoxepin relieves the insomnia.

### Example 38

A patient suffers from a sleep disorder. The patient is prescribed a mixture of E and Z isomers of desmethyldoxepin containing greater than 25% Z isomer in a daily dosage of 5 milligrams prior to bedtime. Administration of the isomers of desmethyldoxepin relieves the insomnia.

### Example 39

A patient suffers from a sleep disorder. The patient is prescribed a mixture of E and Z isomers of desmethyldoxepin containing greater than 50% Z isomer in a daily dosage of 2 milligrams prior to bedtime. Administration of the isomers of desmethyldoxepin relieves the insomnia.

### Example 40

A patient suffers from a sleep disorder. The patient is prescribed a mixture of E and Z isomers of desmethyldoxepin containing greater than 50% Z isomer in a daily dosage of 5 milligrams prior to bedtime. Administration of the isomers of desmethyldoxepin relieves the insomnia.

### Example 41

A patient suffers from a sleep disorder. The patient is prescribed a mixture of E and Z isomers of desmethyldoxepin containing greater than 75% Z isomer in a daily dosage of 5 milligrams prior to bedtime. Administration of the isomers of desmethyldoxepin relieves the insomnia.

Many modifications and variations of the embodiments described herein may be made without departing from the scope, as is apparent to those skilled in the art. The specific embodiments described herein are offered by way of example only.

1. A method for treating insomnia comprising administering to a patient desmethyldoxepin, a pharmaceutically acceptable salt thereof, or a prodrug thereof other than doxepin in a daily dosage ranging from about 0.01 to about 500 milligrams.

2. The method of claim 1, wherein the pharmaceutically acceptable salt of desmethyldoxepin is the hydrochloride salt thereof.

3. The method of claim 1, wherein the prodrug of desmethyldoxepin is a prodrug ester or amide.

4. The method of claim 1, wherein the daily dosage is about 20 to about 300 milligrams.

5. The method of claim 1, wherein the daily dosage is about 0.1 to about 20 milligrams.

6. (canceled)

7. (canceled)

8. The method of claim 1, wherein the insomnia is a chronic insomnia or a non-chronic insomnia.

9. The method of claim 8, wherein the non-chronic insomnia is a transient or a short term insomnia.

10. The method of claim 1, wherein the insomnia is selected from the group consisting of onset insomnia and maintenance insomnia.

11. (canceled)

12. (canceled)

13. The method of claim 1, wherein the desmethyldoxepin, the salt or the prodrug is administered in a cis-(Z) to trans-(E) isomer ratio of about 99:1, 98:2, 95:5, 90:10, 80:20, 70:30, 60:40, 50:50, 40:60, 30:70, 20:80, 10:90, 5:95, 2:98 or 1:99.

14. (canceled)

15. (canceled)

16. (canceled)

17. (canceled)

18. (canceled)

19. (canceled)

20. (canceled)

21. (canceled)

22. (canceled)

23. (canceled)

24. A composition comprising desmethyldoxepin, a pharmaceutically acceptable salt thereof, or a prodrug of desmethyldoxepin other than doxepin in an amount ranging from about 0.01 to about 500 milligrams.

25. The composition of claim 24, wherein the amount is about 20 to about 300 milligrams.

26. The composition of claim 24, wherein the amount is about 0.1 to about 20 milligrams.

27. The composition of claim 24, wherein the amount is about 0.1 to about 10 milligrams.

28. The composition of claim 24, wherein the amount is about 0.1 to about 5 milligrams.

29. The composition of claim 24, wherein the desmethyldoxepin, salt or prodrug is in a cis-(Z) to trans-(E) isomer ratio of about 99:1, 98:2, 95:5, 90:10, 80:20, 70:30, 60:40, 50:50, 40:60, 30:70, 20:80, 10:90, 5:95, 2:98 or 1:99.

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. A method for treating insomnia comprising administering to a patient an isomer of desmethyldoxepin, a pharmaceutically acceptable salt thereof, or a prodrug thereof other than doxepin in a daily dosage ranging from about 0.01 to about 500 milligrams.

47. (canceled)

48. (canceled)

49. (canceled)

50. (canceled)

51. (canceled)

52. (canceled)

53. The method of claim 46, wherein the isomer of desmethyldoxepin, the salt or the prodrug is administered in a cis-(Z) to trans-(E) isomer ratio of about 99:1, 98:2, 95:5, 90:10, 80:20, 70:30, 60:40, 50:50, 40:60, 30:70, 20:80, 10:90, 5:95, 2:98 or 1:99.

54. (canceled)

55. (canceled)

56. The method of claim 46, wherein the insomnia is a chronic insomnia or a non-chronic insomnia.

57. The method of claim 56, wherein the non-chronic insomnia is a transient or a short term insomnia.

58. The method of claim 46, wherein the insomnia is selected from the group consisting of onset insomnia and maintenance insomnia.

59-76. (canceled)
